(19) United States
(12) Patent Application Publication
(10) Pub. No.: US 2026/0232649 A1
(43) Pub. Date: Aug. 13, 2026

(54) AMLODIPINE FREEZE-DRIED COMPOSITIONS AND USES THEREOF

(71) Applicant: Brillian Pharma Inc., Edison, NJ (US)

(72) Inventor: Nuo Wang, Newtown, PA (US)

(73) Assignee: Brillian Pharma Inc., Edison, NJ (US)

(21) Appl. No.: 19/432,512

(22) Filed: Dec. 24, 2025

Related U.S. Application Data
(63) Continuation of application No. 19/153,536, filed on Nov. 13, 2023.
(60) Provisional application No. 63/483,083, filed on Feb. 3, 2023.

(51) Int. Cl. A61K 31/455; A61K 9/00; A61K 9/08; A61K 9/19

(52) U.S. Cl. CPC A61K 31/455; A61K 9/0053; A61K 9/08; A61K 9/19

(57) ABSTRACT

This disclosure provides a novel freeze-dried amlodipine composition which when admixed with an aqueous diluent almost instantly forms an oral solution at the time of use. The disclosed freeze-dried amlodipine composition is stable at room temperature for at least 12-24 months and does not require refrigeration during its shipping, storage, use, and during its shelf-life. In addition, the disclosed freeze-dried amlodipine composition does not contain any alcohol and/or benzoates that are harmful to vulnerable patient populations such as younger children (e.g., children less than six years of age, including infants and neonates). Moreover, the oral solution reconstituted from the disclosed freeze-dried amlodipine composition does not have a potential dose uniformity risk caused by drug sedimentation of an amlodipine suspension.

### CROSS-REFERENCE TO RELATED APPLICATIONS

This application is a continuation of U.S. patent application Ser. No. 19/153,536, filed on Aug. 4, 2025, which is the U.S. national phase of International Patent Application No. PCT/US23/79462, filed on Nov. 13, 2023, which claims priority under 35 U.S.C. § 119 (e) to U.S. Provisional Patent Application No. 63/483,083, filed Feb. 3, 2023. The foregoing applications are incorporated by reference herein in their entirety.

### FIELD OF THE INVENTION

This invention relates generally to freeze-dried amlodipine compositions for oral solution, methods of preparation thereof, and uses thereof.

### BACKGROUND OF THE INVENTION

Amlodipine is used to treat hypertension in adults and children of 6 years and older to lower blood pressure. It was formulated as a tablet dosage form in 2.5, 5, and 10 mg strengths and marketed under the brand name: Norvasc®. Despite its wide use, the tablet form of amlodipine is not well accepted by young children and elderly populations who have difficulty swallowing tablets.

Amlodipine is the choice of calcium channel antagonist for very young children because it can be made into a liquid form without compromising its long duration of action. Most compounded amlodipine suspensions are prepared in a pharmacy by grinding the amlodipine tablets into powder and mixed with a dispersing medium. The compounded suspensions must be refrigerated and have a short shelf life. Several liquid amlodipine formulations are available on the market. However, all the existing amlodipine liquid products have attributes that can be problematic in their use. For example, all of these products except Norliqva® require to be stored under refrigerated (e.g., at 2° C. to 8° C.) conditions during shipment, storage, and use. Maintaining medication under refrigerated conditions throughout the supply chain storage can be challenging. The increasing trend of filling and shipping prescriptions by mail-order pharmacies increases the supply chain risk for medications requiring cold chain shipping. Any breaks in the cold chain supply chain can potentially compromise the effectiveness and sometimes safety of the medication. Also, patients must continue to store received medicines throughout the in-use period to maintain their potency. Amlodipine treatment requires long-term continued daily doses to lower and maintain the blood pressure to a desired range. The refrigeration requirement of these amlodipine liquid products causes particular inconvenience for patients. The risk of temperature excursion outside the specified storage conditions increases for reasons such as forgetting to return the drug product to refrigeration and lack of temperature protection while traveling. Any of these scenarios may result in under-potent products and increase the risk of treatment failure.

In addition, all suspension dosage forms have some tendency to sediment. The settled particles may cause inaccurate dosing, compromising treatment effectiveness or undesirable adverse effects. For example, Katerzia®, a suspension dosage form, tends to have drug particles on the bottom of its container, which may cause uniformity problems during dose division and potentially compromise the treatment outcome.

Further, all of these products contain excipients that may not be suitable for younger children, such as children less than six years of age, including infants and neonates. For example, Norliqva® oral solution contains 4% v/v ethanol. Co-administration of ethanol may alter the drug absorption or metabolism or result in drug interaction. Ethanol may also cause central nervous system adverse effects and gastrointestinal upset in younger children. All other products contain preservatives such as benzoates that may not be as safe for pediatric use, although preservatives may be needed for controlling mold, inhibiting yeast growth, and protecting from bacterial propagation.

Therefore, there is a strong need for an improved amlodipine composition for younger children and elderly patients.

### SUMMARY OF THE INVENTION

This disclosure addresses the need mentioned above in a number of aspects. In one aspect, this disclosure provides a novel freeze-dried amlodipine composition, which when admixed with an aqueous diluent, almost instantly forms an oral solution at the time of use. The disclosed freeze-dried amlodipine composition does not require refrigeration in shipping, storage, and during treatment. It does not contain harmful ingredients such as benzoates and ethanol that are not suitable for younger children.

In some embodiments, the freeze-dried amlodipine composition comprises at least one therapeutically effective amount of amlodipine salt and at least one pharmaceutically acceptable excipient, wherein the composition is adapted to form an oral solution when admixed with an aqueous medium, wherein the composition remains stable at 20-25° C. for a storage period of at least 12-24 months, and wherein the composition comprises 1.0% w/w or less of 3-ethyl 5-methyl [2-(2-aminoethoxymethyl)-4-(2-chlorophenyl)-6-methyl-3,5-pyridinedicarboxylate fumarate at the end of the 12-24 months storage period.

In some embodiments, the oral solution containing 5 mg equivalent of amlodipine base following a single oral administration of the oral solution to adult subjects under fasted conditions provides the same pharmacokinetic profile as a tablet containing 5 mg equivalent of amlodipine base, wherein the pharmacokinetic profile has an area under the curve of from about 83.5 ng·hr/mL to about 256.5 ng·hr/mL and a Cmax of from about 1.8 ng/ml to about 5 ng/ml.

In some embodiments, the composition is capable of dispersing and dissolving within less than 60 seconds upon admixing with the aqueous medium. In some embodiments, the composition is capable of dispersing and dissolving within less than 60 seconds upon admixing with the aqueous medium, after storage at 20-25° C. for a period of at least 12-24 months.

In some embodiments, the composition is in the form of power, granules, agglomerates, or power cake. In some embodiments, the composition is provided as one or more unit doses.

In some embodiments, the composition comprises from about 0.035% wt/wt to about 99.64% wt/wt of the amlodipine salt.

In some embodiments, the oral solution reconstituted from the freeze-dried composition comprises from about 0.0087% w/w to about 10.52% w/w of the amlodipine salt.

In some embodiments, the composition comprises from about 0.0005% w/w to about 99.96% w/w of the excipient.

In some embodiments, the excipient comprises a bulking agent. In some embodiments, the composition comprises from about 0% w/w to about 99.96% w/w of the bulking agent. In some embodiments, the oral solution comprises from about 0% w/w to about 97.1% w/w of the bulking agent.

In some embodiments, the composition comprises a taste-masking agent. In some embodiments, the composition comprises from about 0.0005% w/w to about 99.8% w/w of the taste-masking agent. In some embodiments, the oral solution comprises from about 0.000125% w/w to about 43.13% w/w of the taste-masking agent.

In some embodiments, the composition comprises: about 5% w/w to about 8% w/w of the amlodipine salt, about 91% w/w to about 94% w/w of the bulking agent, and about 1% w/w to about 3% w/w of the taste-masking agent.

In some embodiments, the oral solution comprises: from about 0.068% w/w to about 0.26% w/w of the amlodipine salt, from about 1.234% w/w to about 4.75% w/w of the bulking agent, from about 0.0039% w/w to about 0.015% w/w of the taste-masking agent, and from about 94.97% w/w to about 98.69% w/w of water.

In some embodiments, the aqueous medium comprises water, juice, a buffer, a solution, or a combination thereof. In some embodiments, the aqueous medium comprises a sweetened water solution, a flavored water solution, or a combination thereof. In some embodiments, the aqueous medium has a volume of from about 0.3 mL to about 30 mL per unit dose of amlodipine base.

In some embodiments, the composition further comprises a coloring agent, an antioxidant, a pH-adjusting agent, an antifoaming agent, or a combination thereof. In some embodiments, the aqueous medium is free of an organic solvent, such as ethanol. In some embodiments, the aqueous medium is free of a preservative or a harmful preservative. A harmful preservative refers to any preservative that may produce any unwanted side effects in human subjects or a specific population of human subjects such as infants and neonates.

In some embodiments, the oral solution is adapted for oral administration to a human subject. In some embodiments, the human subject has difficulty swallowing a rigid-shaped solid pharmaceutical dosage form, such as capsules or tablets.

In some embodiments, the composition is prepared by in situ freeze-drying a liquid solution of the composition in the container.

In another aspect, this disclosure provides an oral solution for oral administration prepared by reconstituting from the composition or the unit dose, as described herein.

In another aspect, this disclosure provides a unit dose comprising the composition described herein, and a product comprising the composition or the unit dose, as described herein.

In another aspect, this disclosure also provides a method for forming the composition or the unit dose, as described herein. In some embodiments, the method comprises: dissolving a therapeutically effective amount of an amlodipine salt and at least one pharmaceutically acceptable excipient to yield a solution containing from about 0.0083% w/w to 10.52% w/w of the amlodipine salt; filling the solution of a single dose of the amlodipine salt into a container; freeze-drying the solution to form a freeze-dried composition containing the amlodipine salt inside the container; and sealing the container.

In yet another aspect, this disclosure further provides a method of treating hypertension or coronary artery disease (CAD) in a subject in need thereof. In some embodiments, the method comprises: reconstituting the composition described herein with an aqueous medium to form an amlodipine oral solution at the time of administration, and orally administering to the subject the oral solution comprising a therapeutically effective amount of amlodipine.

In some embodiments, the therapeutically effective amount of amlodipine administered produces a pharmacokinetic profile bioequivalent to that of an amlodipine 5 mg tablet, such as a Norvasc® 5 mg tablet, in adult subjects under fasted conditions.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### DETAILED DESCRIPTION OF THE INVENTION

This disclosure provides a novel freeze-dried amlodipine composition which when admixed with an aqueous diluent almost instantly forms an oral solution at the time of use. The disclosed freeze-dried amlodipine composition is stable at room temperature for at least 12-24 months based on extrapolation of stability performance of the composition stored at an accelerated condition of 40° C.±2° C./75% RH±5% RH (according to International Council for Harmonization (ICH) guideline Q1A, Stability Testing of New Drug Substances and Products) for 6 months. It does not require refrigeration during its shipping, storage, use, and during its shelf-life. In addition, the disclosed freeze-dried amlodipine composition does not contain any alcohol and/or preservatives such as benzoates that may be harmful to vulnerable patient populations such as younger children (e.g., children less than six years of age, including infants and neonates). Moreover, the disclosed freeze-dried amlodipine composition does not have a potential dose uniformity risk caused by drug sedimentation as in existing amlodipine liquid formulations.

### Freeze-Dried Amlodipine Compositions

In some embodiments, the freeze-dried amlodipine composition for oral solution may include at least one therapeutically effective amount of amlodipine salt and at least one pharmaceutically acceptable excipient. The freeze-dried amlodipine composition for oral solution is useful for treating hypertension and coronary artery diseases (CAD). The freeze-dried amlodipine composition for oral solution provides an advantage over traditional solid dosage forms (i.e., tablets, capsules, etc.) for hypertension and CAD patients who have difficulty swallowing such solid dosage forms. The patients with difficulty swallowing such solid dosage forms are usually young children still developing the ability to swallow solid dosage forms, or senior patients with additional medical conditions such as stroke, Parkinson's, and Alzheimer's diseases that adversely affect their normal swallow function. The freeze-dried amlodipine composition for oral solution can be almost instantly converted to an oral solution at the time of dosing, providing an easily ingestible liquid dosage form of amlodipine for young children and elderly patients.

Due to in dry state before dosing, the freeze-dried amlodipine composition also provides several advantages over the currently available oral liquid dosage forms (e.g., Katerzia® oral suspension). For example, the freeze-dried amlodipine composition is stable for a storage period of at least 12-24 months, thus eliminating the requirement of storing the product under refrigerator conditions during shipping, storage, and use. The refrigerator condition refers to the temperature and storage definitions described in United States Pharmacopeia (USP). The refrigerator is defined as a cold place in which the temperature is controlled between 2° C. and 8° C. The elimination of refrigeration requirements provides several benefits for the logistics and use of the product. It reduces the risk of any product quality deterioration due to temperature excursion. It also reduces the logistic cost for shipping, storage, and use. Furthermore, an amlodipine product without the need for refrigeration is more convenient for patients to use, especially during travel, and therefore increases patient compliance.

The freeze-dried amlodipine composition for an oral solution does not contain any ingredients such as ethanol (e.g., an ingredient in Norliqva® oral solution) or sodium benzoate (e.g., an ingredient in Katerzia® oral suspension) that are potentially harmful to younger children less than 6 years of age.

In addition, the disclosed freeze-dried amlodipine composition has the following beneficial characteristics.

### Stability Characteristics

The freeze-dried amlodipine composition for oral solution possesses superior stability characteristics. In some embodiments, the composition is storage stable at 15°-25° C. (59°-77° F.), such as 20°-25° C. (68°-77° F.), for at least 24 months, extrapolated from the stability performance of the composition stored at 40±2° C. (75% relative humidity) for at least 6 months.

In some embodiments, the freeze-dried amlodipine composition has a minimal amount of impurity after an extended period of storage at 15°-25° C. (59°-77° F.), such as 20°-25° C. (68°-77° F.), for at least 12-24 months (e.g., at least 12, 13, 14, 15, 16, 17, 18, 19, 20, 21, 22, 23, 24 months) extrapolated from the amount of impurity after storage at 40±2° C. (75% relative humidity (RH)) for at least 6 months. In some embodiments, the impurity may include 3-Ethyl 5-methyl[2-(2-aminoethoxymethyl)-4-(2-chlorophenyl)-6-methyl-3,5-pyridinedicarboxylate] fumarate (also referred to as “amlodipine-related compound A” in United States Pharmacopeia), any unspecified degradation product, and/or total degradation products. In some embodiments, the composition contains no more than 1.0% of amlodipine-related compound A, no more than 0.2% of any unspecified degradation product, and no more than 1.5% of total degradation products at the end of storage at 40±2° C. (75% RH) for 6 months. In some embodiments, the composition contains no more than 1.0% of 3 amlodipine-related compound A, no more than 0.2% of any unspecified degradation product, and no more than 1.5% of total degradation products as defined in the monograph after storage at about 15°-25° C. (59°-77° F.), such as at about 20°-25° C. (68°-77° F.), for at least 12-24 months (e.g., at least 12, 13, 14, 15, 16, 17, 18, 19, 20, 21, 22, 23, 24 months), extrapolated from the amount of impurity after storage at 40±2° C. (75% relative humidity (RH)) for at least 6 months.

### Quick Reconstitution Characteristics

In some embodiments, the freeze-dried composition can be in power, granules, agglomerates, power cake, or any other forms resulting from a freeze-drying process. One aspect of these freeze-dried compositions is their fast dispersing and dissolving characteristics upon admixing with an aqueous diluent (also referred to as aqueous medium). The freeze-dried composition dissolves almost instantly when admixed with an aqueous diluent to form a solution suitable for oral administration to a subject, such as a human. As used herein, “almost instantly” refers to the short time required to reconstitute the freeze-dried composition to form an oral solution. In some embodiments, the reconstitution time is less than 60 seconds. In some embodiments, the reconstitution time is less than 45 seconds. In some embodiments, the reconstitution time is less than 30 seconds. In some embodiments, the reconstitution time is less than 15 seconds.

In some embodiments, the freeze-dried amlodipine composition is capable of dispersing and dissolving almost instantly (e.g., less than 60, 50, 40, 30, 20, or 10 seconds) in an aqueous diluent to form an oral solution (e.g., clear oral solution), when mixed with the aqueous diluent. In some embodiments, the composition disperses/dissolves in an aqueous diluent (e.g., water) and becomes a clear solution in less than 60 seconds when mixed with the aqueous diluent. In some embodiments, the composition disperses/dissolves in an aqueous diluent and becomes a clear solution in less than 45 seconds when mixed with the aqueous diluent. In some embodiments, the composition disperses/dissolves in an aqueous diluent and becomes a clear solution in less than 30 seconds when mixed with the aqueous diluent. In some embodiments, the composition disperses/dissolves in an aqueous diluent and becomes a clear solution in less than 15 seconds when mixed with the aqueous diluent.

In some embodiments, the freeze-dried amlodipine composition for oral solution is packaged in unit-dose containers that contain an amlodipine salt in an amount corresponding to 2.5 mg, 5 mg, or 10 mg amlodipine free base. These unit-dose packaged freeze-dried amlodipine compositions dissolve and become a clear solution in less than 60 seconds when mixed with 5-10 mL of an aqueous diluent. In some embodiments, these unit-dose packaged freeze-dried amlodipine compositions dissolve and become a clear solution in less than 45 seconds when mixed with 5-10 mL of an aqueous diluent. In some embodiments, these unit-dose packaged freeze-dried amlodipine compositions dissolve and become a clear solution in less than 30 seconds when mixed with 5-10 mL of an aqueous diluent. In some embodiments, these unit-dose packaged freeze-dried amlodipine compositions dissolve and become a clear solution in less than 15 seconds when mixed with 5-10 mL of an aqueous diluent.

In some embodiments, the freeze-dried amlodipine composition for oral solution retains its fast reconstitution characteristics during storage. In some embodiments, the freeze-dried amlodipine composition for oral solution retains its fast reconstitution characteristics after storage at 40±2° C. (75% relative humidity) for at least 6 months. In some embodiments, the freeze-dried amlodipine composition for oral solution retains its fast reconstitution characteristics after storage at about 15°-25° C. (59°-77° F.), such as at about 20°-25° C. (68°-77° F.), for at least 12-24 months (e.g., at least 12, 13, 14, 15, 16, 17, 18, 19, 20, 21, 22, 23, 24 months).

### Pharmacokinetic (PK) Profiles

In some embodiments, the oral solution reconstituted from the freeze-dried amlodipine composition produces certain pharmacokinetic (PK) profiles when administered to adult subjects. As used herein, the term “pharmacokinetic profile” or “PK profile” refers to a bodily concentration of a pharmaceutically active agent, or a metabolite thereof (e.g., an active metabolite), namely, a concentration of the agent or a metabolite thereof in a physiological system of an organism (whole body, blood, plasma, lymph, tissue, organ, and the likes) to which the compound has been administered, as a function of time. Typically, a PK profile is considered from a time point of administration of the compound to a time point at which the compound is no longer detectable in the organism or to any intermediate period of time between administration of the compound and a time at which it is no longer detectable in the organism (e.g., due to excretion); hence, a PK profile describes the bodily concentration in a specific physiological system of a specific compound between administration and dissipation, as affected by the mechanisms of liberation, absorption, distribution, metabolism and excretion/secretion of the compound. Since each organism, and each individual organism within a genus of an organism, reacts differently to the administration of the agent, a PK profile may be different, and in some cases highly variable from subject to subject, and may be different within an individual subject based on a current physiological state, medical condition, environmental conditions and even the time of day.

In some embodiments, the oral solution reconstituted from the freeze-dried amlodipine composition containing 5 mg base equivalent amlodipine besylate exhibits the following pharmacokinetic parameters when administered to healthy adult subjects under fasting conditions:
Peak Plasma Concentration (measured by Cₘₐₓ): ranging from 1.8 ng/ml to 5.0 ng/ml;
Total amount of amlodipine absorbed at time=144 hours post dosing (measured by area under the curve AUC₀₋ₜ): ranging from 79.7 to 230.0 h*ng/ml; and/or
Total amount of amlodipine absorbed at time=∞ hours post dosing (measured by area under the curve AUC_{0-∞}): ranging from 83.5 to 256.5 h*ng/mL.

As used herein, the term “area under the plasma concentration-time curve” or “AUC” is related to the total amount of an active measurable in the systemic circulation following administration of a single dose. The AUC is a mathematical and visual representation of the aggregate amount of the active in the systemic circulation over a given period of time. Changes in the AUC need not necessarily reflect changes in the total amount of the active absorbed but can reflect modifications in the kinetics of distribution, metabolism, and excretion.

In some embodiments, the oral solution reconstituted from the freeze-dried amlodipine composition containing 5 mg base equivalent amlodipine besylate exhibits a pharmacokinetic profile that is bioequivalent to that of Norvasc® tablet 5 mg. Norvasc® tablet is a commercial product manufactured by Pfizer Pharmaceuticals LLC, a division of Pfizer Inc. As used herein, the term “bioequivalent” is defined in the Code of Federal Regulations Title 21 as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” The absence of a significant difference in the rate and extent of amlodipine absorbed between the oral solution reconstituted by the freeze-dried amlodipine composition and Norvasc® tablet is established by the calculation of a 90% confidence interval for the ratio of the averages (population geometric means) of the measures (i.e., Cₘₐₓ, AUC₀₋ₜ, and AUC_{0-∞}) between the oral solution reconstituted by the freeze-dried amlodipine composition and Norvasc® tablet and the calculated confidence interval should fall within 80-125% for the ratio of the averages.

When treating hypertension or CAD, the oral solution reconstituted from the freeze-dried amlodipine composition can be administered with or without food. The oral solution reconstituted from the freeze-dried amlodipine composition produces bioequivalent pharmacokinetic profiles (i.e., Cₘₐₓ, AUC₀₋ₜ, and AUC_{0-∞}) when administered with or without food. The 90% confidence interval for the ratio of population geometric means between fed and fasted administration is within the limits of 80-125%, and therefore is absent of a food effect. As used herein, the term “food effect” refers to a relative difference in AUC (area under the curve), Cₘₐₓ (maximum plasma concentration), and/or Tₘₐₓ (time to maximum concentration) of an active substance, when a substance or a composition thereof, such as a tablet, a capsule or a liquid, is administered orally to a subject concomitantly with food or in a fed state as compared to the same values when the same composition is administered in a fasted state.

In some embodiments, the composition may include from about 0.035% w/w to about 99.64% w/w (e.g., 0.035, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.64% w/w) of the amlodipine salt.

In some embodiments, the oral solution may include from about 0.0087% w/w to about 10.52% w/w (e.g., 0.0087, 0.001, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.52% w/w) of the amlodipine salt.

In some embodiments, the composition may include from about 0.0005% w/w to about 99.96% w/w (e.g., 0.0005, 0.001, 0.01, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.96% w/w) of the excipient.

In some embodiments, the excipient may include a bulking agent. In some embodiments, the composition may include from about 0% w/w to about 99.96% w/w (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.96% w/w) of the bulking agent.

In some embodiments, the oral solution may include from about 0% w/w to about 97.1% w/w (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 97.1% w/w) of the bulking agent.

In some embodiments, the excipient may include a taste-masking agent. In some embodiments, the composition may include from about 0.0005% w/w to about 99.8% w/w (e.g., 0.0005, 0.001, 0.01, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.8% w/w) of the taste-masking agent.

In some embodiments, the oral solution may include from about 0.000125% w/w to about 43.13% w/w (e.g., 0.000125, 0.0005, 0.001, 0.01, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 43.13% w/w) of the taste-masking agent.

In some embodiments, the composition may include: about 5% w/w to about 7% w/w of the amlodipine salt, about 91% w/w to about 95.9% w/w of the bulking agent, and about 0.1% w/w to about 2% w/w of the taste-masking agent.

In some embodiments, the oral solution may include: from about 0.068% w/w to about 0.26% w/w (e.g., 0.068, 0.073, 0.078, 0.083, 0.088, 0.093, 0.1, 0.15, 0.2, 0.25, 0.26% w/w) of the amlodipine salt, from about 1.234% w/w to about 4.75% w/w (e.g., 1.234, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.75% w/w) of the bulking agent, from about 0.0039% w/w to about 0.015% w/w (e.g., 0.0044, 0.0049, 0.0054, 0.0059, 0.0064, 0.0069, 0.0074, 0.0079, 0.0084, 0.0089, 0.0094, 0.0099, 0.0104, 0.0109, 0.0114, 0.0119, 0.0124, 0.0129, 0.0134, 0.0139, 0.0144, 0.0149, 0.015% w/w) of the taste-masking agent, and from about 94.97% to about 98.69% w/w (e.g., 94.97, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.69% w/w) of water.

In some embodiments, the freeze-dried composition, when admixed with an aqueous medium, almost instantly forms an oral solution. In some embodiments, the composition may include at least one therapeutically effective amount of amlodipine salt and at least one pharmaceutically acceptable excipient, wherein the composition is stable at 20-25° C. for a storage period of at least 12-24 months, wherein the composition has 1.0% or less of amlodipine-related compound A at the end of the 12-24 months storage period, and wherein the oral solution provides a pharmacokinetic profile in which amlodipine has an area under the curve (AUC_{0-∞}) of about 83.5 ng*hr/mL to about 256.5 ng*hr/mL and a Cₘₐₓ of about 1.8 ng/ml to about 5 ng/ml following a single oral administration of the oral solution to adult subjects under fasted conditions at a dose equivalent to 5 mg of amlodipine (e.g., Norliqva® table 5 mg).

In some embodiments, the freeze-dried composition, when admixed with an aqueous medium, almost instantly forms an oral solution. In some embodiments, the composition may include at least one therapeutically effective amount of amlodipine salt and at least one pharmaceutically acceptable excipient, wherein the dry composition is stable at 20-25° C. for a storage period of at least 12-24 months, wherein the stable dry composition has 1.0% or less of amlodipine-related compound A at the end of the 12-24 months storage period, and wherein the oral solution provides a bioequivalent pharmacokinetic profile to that of the Norvasc tablet following a single oral administration of the oral solution or Norvasc® tablet to adult subjects under fasted conditions at a dose equivalent to 5 mg of amlodipine.

### Active Pharmaceutical Ingredients (APIs)

The freeze-dried amlodipine composition for oral solution contains at least one amlodipine compound as a pharmaceutically active ingredient(s). Amlodipine has a chemical structure as follows:

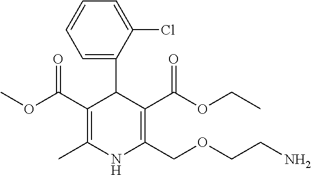 [SMILES: CCOC(=O)C1=C(COCCN)NC(C)=C(C(=O)OC)C1c1ccccc1Cl]

The amlodipine compound can be in any of its salt forms. In some embodiments, amlodipine salts refer to any amlodipine salt formed by combining the ionized amlodipine with a counter-ion acid. In some embodiments, the amlodipine salt is amlodipine besylate. In some embodiments, the amlodipine salt is selected from amlodipine tosylate, amlodipine mesylate, amlodipine succinate, amlodipine salicylate, amlodipine maleate, amlodipine acetate, amlodipine hydrochloride, amlodipine benzoate, amlodipine malate, amlodipine fumarate, amlodipine adipate, amlodipine camsylate, amlodipine nicotinate, or a mixture thereof.

In some embodiments, the amlodipine active base or salts can be in amorphous form. In some embodiments, the amlodipine salts can be in any available crystal form. In some embodiments, amlodipine salts can be in an anhydrous form. In some embodiments, the amlodipine salts can be in a hydrate form.

As used herein, the term “active,” “active ingredient,” “active agent,” or “pharmaceutically active ingredient” refers to a chemical entity intended to furnish pharmacological activity or to otherwise have a direct effect in the diagnosis, cure, mitigation, treatment or prevention of disease, or to have direct effects in restoring, correcting or modifying physiological functions in a subject.

In some embodiments, the amount of amlodipine salt in the freeze-dried composition may range from about 0.035% w/w to about 99.64% w/w. In some embodiments, the amount of the amlodipine active (e.g., free base equivalent) in the freeze-dried composition may range from about 0.043% w/w to about 99% w/w, from about 0.057% w/w to about 80% w/w, from about 0.086% w/w to about 70% w/w, from about 0.173% w/w to about 60% w/w, from about 0.346% w/w to about 50% w/w, from about 0.461% w/w to about 40% w/w, from about 0.553% w/w to about 30% w/w, from about 0.689% w/w to about 20% w/w, from about 1.36% w/w to about 10% w/w, or any intermediate range thereof.

In some embodiments, each daily therapeutic dose of the freeze-dried composition contains an amount of amlodipine salt equivalent to 1.25 mg to 20 mg (e.g., 1.25, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 mg) of amlodipine base for treating hypertension or CAD in a patient.

The freeze-dried composition for an oral solution can be almost instantly converted to an amlodipine oral solution when in contact with a suitable amount of aqueous liquid (e.g., water) and without the need for any vigorous mixing or shaking. Once reconstituted, the amount of the amlodipine active (free base equivalent) in the reconstituted oral solution ranges from about 0.0087% w/w to about 10.52% w/w. In some embodiments, the amount of the amlodipine active may range from about 0.0087% w/w to about 0.3% w/w, about 0.3% w/w to about 0.6% w/w, about 0.6% w/w to about 0.9% w/w, about 0.9% w/w to about 1.2% w/w, about 1.2% w/w to about 1.5% w/w, about 1.5% w/w to about 1.8% w/w, about 1.8% w/w to about 2.1% w/w, about 2.1% w/w to about 2.4% w/w, about 2.4% w/w to about 2.7% w/w, about 2.7% w/w to about 3% w/w, about 3% w/w to about 3.3% w/w, about 3.3% w/w to about 3.6% w/w, about 3.6% w/w to about 3.9% w/w, about 3.9% w/w to about 4.2% w/w, about 4.2% w/w to about 4.5% w/w, about 4.5% w/w to about 4.8% w/w, about 4.8% w/w to about 5.1% w/w, about 5.1% w/w to about 5.4% w/w, about 5.4% w/w to about 5.7% w/w, about 5.7% w/w to about 6% w/w, about 6% w/w to about 6.3% w/w, about 6.3% w/w to about 6.6% w/w, about 6.6% w/w to about 6.9% w/w, about 6.9% w/w to about 7.2% w/w, about 7.2% w/w to about 7.5% w/w, about 7.5% w/w to about 7.8% w/w, about 7.8% w/w to about 8.1% w/w, about 8.1% w/w to about 8.4% w/w, about 8.4% w/w to about 8.7% w/w, about 8.7% w/w to about 9% w/w, about 9% w/w to about 9.3% w/w, about 9.3% w/w to about 9.6% w/w, about 9.6% w/w to about 9.9% w/w, about 9.9% w/w to about 10.2% w/w, or about 10.2% w/w to about 10.52% w/w, or any intermediate range thereof.

### Excipients

### Bulking Agent/Filler

In some embodiments, the freeze-dried composition may include a bulking agent as an excipient. Bulking agents serve to either bulk up the volume or facilitate formation of the quick reconstitution characteristics of the freeze-dried composition. The reconstitution refers to the process of complete dissolution of the freeze-dried composition when mixing with a diluent. In some embodiments, the bulking agents increase the volume of the freeze-dried composition so that the production of the composition becomes feasible on common pharmaceutical processing equipment. In some embodiments, the bulking agents facilitate formation of the quick reconstitution characteristics of the freeze-dried composition so that an amlodipine oral solution can be formed very quickly by reconstitution when the freeze-dried composition is mixed with an aqueous diluent. In some embodiments, such reconstitution does not require vigorous mixing, stirring, or shaking. In some embodiments, the time for quick reconstitution is less than 60 seconds. In some embodiments, the time for quick reconstitution is less than 45 seconds. In some embodiments, the time for quick reconstitution is less than 30 seconds. In some embodiments, the time for quick reconstitution is less than 15 seconds.

A bulking agent can be any aqueous soluble pharmaceutically acceptable ingredients that are chemically compatible with amlodipine salts and enhance the quick reconstitution characteristics of the freeze-dried composition for oral solution. Examples of suitable bulking agents include mannitol, glycine, lactose, sucrose, glucose, sorbitol, xylitol, trehalose, raffinose, histidine, arginine, glycine, dextran, povidone, or a combination thereof.

In some embodiments, the bulking agent contained in the freeze-dried composition for oral solution may range from: about 0.0005% to about 99.96% w/w, such as about 1% to about 99.0% w/w, about 10% to about 98% w/w, about 20% to about 97% w/w, about 30% to about 96% w/w, about 40% to about 95% w/w, or any intermediate range thereof.

In some embodiments, the bulking agent in a reconstituted amlodipine oral solution may range from: about 0% w/w to about 99.8% w/w, such as about 0% w/w to about 1% w/w, about 1% w/w to about 5% w/w, about 5% w/w to about 10% w/w, about 10% w/w to about 15% w/w, about 15% w/w to about 20% w/w, about 20% w/w to about 25% w/w, about 25% w/w to about 30% w/w, about 30% w/w to about 35% w/w, about 35% w/w to about 40% w/w, about 40% w/w to about 45% w/w, about 45% w/w to about 50% w/w, about 50% w/w to about 55% w/w, about 55% w/w to about 60% w/w, about 60% w/w to about 65% w/w, about 65% w/w to about 70% w/w, about 70% w/w to about 75% w/w, about 75% w/w to about 80% w/w, about 80% w/w to about 85% w/w, about 85% w/w to about 90% w/w, about 90% w/w to about 95% w/w, or about 95% w/w to about 99.8% w/w, or any intermediate range thereof.

### Taste-Masking Agents

Amlodipine and its salts may have an unpleasant taste that is not preferred by the subjects being treated. In some embodiments, a taste-masking agent may be added to the freeze-dried composition to improve the taste and flavor of the oral solution reconstituted from the freeze-dried composition. In some embodiments, the taste-masking agents can be a sweetener, a flavoring agent, or any other agents that give the oral solution a palatable taste or smell, or a mixture thereof.

A sweetener is a substance that provides a sweet taste in the mouth of a subject. In some embodiments, a sweetener can be sugar. Sugar is the generic name for sweet-tasting, soluble carbohydrates, many of which are used in food. Simple sugars, also called monosaccharides, may include glucose, fructose, and galactose. Compound sugars, also called disaccharides or double sugars, are molecules made of two monosaccharides joined by a glycosidic bond. Common examples are sucrose (glucose+fructose), lactose (glucose+galactose), and maltose (two molecules of glucose). Table sugar, granulated sugar, and regular sugar refer to sucrose, a disaccharide composed of glucose and fructose. Examples of natural sweeteners may include sucrose, glucose, fructose, D-ribose, galactose, maltose, maltitol, isomalt, mannitol, sorbitol, Xylitol, glycerol, Thaumatin, Glycyrrhizin, stevioside, and erythritol.

A sweetener can also be sugar alcohol. Sugar alcohols (also called polyhydric alcohols, polyalcohols, alditols or glycitols) are organic compounds, typically derived from sugars, containing one hydroxyl group attached to each carbon atom. They are white, water-soluble solids that can occur naturally or be produced industrially by hydrogenation of sugars. Since they contain multiple hydroxyl groups, they are classified as polyols. Examples of sugar alcohols may include xylitol, sorbitol, erythritol, hydrogenated starch hydrolysates (HSH), isomalt, lactitol, maltitol, and mannitol.

A sweetener can also be a sugar substitute or artificial sweetener. A sugar substitute is a substance that provides a sweet taste like that of sugar while with higher intensity and containing significantly less food energy than sugar-based sweeteners. Artificial sweeteners may be derived through manufacturing of plant extracts or processed by chemical synthesis. Examples of artificial sweeteners may include neotame, sucralose, allulose, acesulfame potassium, advantame, alitame, aspartame, aspartame-acesulfame salt, cyclamate, neohesperidin DC, mogrosides, saccharin, steviol glycosides (stevia), and monk fruit.

In some embodiments, the freeze-dried composition for oral solution may include one or more sweeteners. In some embodiments, the freeze-dried composition may include a mixture of sweeteners. In some embodiments, the freeze-dried composition may include neotame.

The amount of sweetener presented in the freeze-dried composition for oral solution should be sufficient to mask the unpleasant taste of amlodipine and provide a sweet taste to the oral solution produced by the freeze-dried composition for oral solution. The amount of sweetener presented in the freeze-dried composition depends on the sweetness intensity of selected sweeteners. For example, a lower amount of sweetener may be used when the sweetener intensity is higher.

In some embodiments, the amount of the taste-masking agent (e.g., sweetener) in the freeze-dried composition for oral solution may range from about 0.0005% w/w to about 99.8% w/w, such as about 0.05% to about 90% w/w, about 0.1% to about 80% w/w, about 1% to about 80% w/w, about 10% to about 70% w/w, about 20% to about 60% w/w, or any intermediate range thereof.

In some embodiments, the amount of the taste-masking agent (e.g., sweetener) in the oral solution reconstituted from the freeze-dried composition for oral solution may range from about 0.000125% w/w to about 43.13% w/w, such as about 0.0002% w/w to about 40% w/w, about 0.001% w/w to about 30% w/w, about 0.01% w/w to about 20% w/w, about 0.1% w/w to about 10% w/w, about 0.5% w/w to about 5% w/w, or any intermediate range thereof.

In some embodiments, the taste-masking agent can be a flavoring agent that can be one of natural or synthetic flavoring agents, such as bananas flavor, grape flavor, cherry, pineapple, raspberry, vanillin flavor, coconut, cinnamon, strawberry, lime, peach, orange, lemon, chocolate flavors, caraway, clove, dill, orange, peppermint, or a combination thereof.

In some embodiments, the taste-masking agent can be a flavor enhancer, such as citric acid, malic acid, tartaric acid, amino acids, monosodium glutamate, table salt/sea salt, or a combination thereof. In some embodiments, the taste-masking agent can be a flavor potentiator which increases the perception of the taste of sweeteners or suppress the taste of bitterness, such as thaumatine, neohesperidine dihydro chalcone, glycyrrhizin, hydroxyflavones, gamma-amino butyric acid, or a combination thereof.

In some embodiments, the taste-masking agent can be a mixture of sweeteners, flavoring agents, flavor enhancers, flavor potentiators, or a combination thereof.

In some embodiments, the freeze-dried composition may include pharmaceutical excipients that will enable the composition to be self-dispersible in an aqueous medium, such as surfactants, highly water-soluble materials, and/or disintegrants.

Surfactants are amphiphilic compounds that lower the interfacial tension between two liquids, between a gas and a liquid, or between a liquid and a solid. When the composition is in contact with an aqueous medium such as water, surfactants act as wetting agents that help the composition to be wetted and promote faster dissolving or dispersion of the composition. The surfactants can also act as emulsifiers to disperse water-insoluble compositions to be dispersed as an emulsion. Surfactants suitable for this application are ionic surfactants and nonionic surfactants. The ionic surfactants include anionic surfactants such as ammonium lauryl sulfate, sodium lauryl sulfate, dioctyl sodium sulfosuccinate, perfluoro octane sulfonate, perfluoro butane sulfonate, alkyl-aryl ether phosphates, alkyl ether phosphates, and sodium stearate; cationic surfactants such as octenidine dihydrochloride, cetrimonium bromide (CTAB), cetylpyridinium chloride (CPC), benzalkonium chloride (BAC), benzethonium chloride (BZT), dimethyldioctadecylammonium chloride, and dioctadecyldimethyl ammonium bromide (DODAB); and zwitterionic surfactants such as phospholipids. The nonionic surfactants include ethoxylates, fatty alcohol ethoxylates, alkylphenol ethoxylates, fatty acid ethoxylates, Ethoxylated amines and/or fatty acid amides, terminally blocked ethoxylates, Fatty acid esters of polyhydroxy compounds, fatty acid esters of glycerols, fatty acid esters of sorbitols (Spans & Tweens).

In some embodiments, highly water-soluble materials are highly hydrophilic and have high solubility in water. These materials include monosaccharide and disaccharide sugars, salts, amino acids, and hydrophilic polymers. Examples of monosaccharide and disaccharide sugars that can be used for this invention are glucose, fructose, galactitol, mannitol, lactose, sucrose, xylose, ribose, mannose, dextrose, maltose, trehalose, sorbitol, xylitol, maltose, glycerol, erythritol, threitol, arabitol, xylitol, ribitol, fucitol, iditol, inositol, volemitol, isomalt, maltitol, lactitol, maltotriitol, maltotetraitol, polyglycitol, and a combination thereof. Highly water-soluble salts may include sodium and potassium salts such as sodium chloride, potassium chloride, and a combination thereof. Highly water-soluble amino acids are charged or polar amino acids. Examples of amino acids may include arginine, lysine, aspartic acid, glutamic acid, histidine, serine, threonine, cysteine, and a combination thereof. Hydrophilic polymers may be natural, modified natural, or synthetic polymers. Examples may include gelatin, Arabic gum, sodium alginate, acacia gum, agarose, xanthan gum, carrageenan, pectin, polyvinyl pyrrolidone, hydroxypropyl methylcellulose, hydroxypropyl cellulose, sodium carboxymethyl cellulose, polyacrylic acids, polyethylene glycols, polyethylene oxides, and a combination thereof.

In another embodiment, a matrix-forming excipient can be added to the solution before subjecting it to the freeze-drying process so that when the freeze-dried cake in the container will maintain its cake shape after freeze-drying and will not crumble in the container before being constituted.

In some embodiments, the composition can further contain additional additives that enable the composition to function safely and effectively. Other inactive ingredients, such as preservatives, coloring agents, antioxidants, antifoaming agents, and pH-adjusting agents, can be added to the solution.

In some embodiments, the dry pharmaceutical composition can also contain a pH-adjusting agent. The pH adjusting agents may be selected from acidic or basic compounds such as acetic acid, adipic acid, ammonium aluminum sulphate, ammonium, bicarbonate, ammonium carbonate, ammonium citrate, dibasic, ammonium citrate, monobasic, ammonium hydroxide, ammonium phosphate, dibasic, ammonium phosphate, monobasic, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, dibasic, calcium phosphate, monobasic, calcium phosphate, tribasic, calcium sulphate, citric acid, fumaric acid, gluconic acid, hydrochloric acid, lactic acid, magnesium carbonate, magnesium citrate, magnesium fumarate, magnesium hydroxide, magnesium oxide, magnesium phosphate, magnesium sulphate, malic acid, phosphoric acid, potassium acid tartrate, potassium aluminum sulphate, potassium bicarbonate, potassium carbonate, potassium chloride, potassium citrate, potassium fumarate, potassium hydroxide, potassium lactate, potassium phosphate, dibasic, potassium phosphate, tribasic, potassium sulphate, sodium acetate, sodium bicarbonate, sodium bisulphate, sodium carbonate, sodium citrate, sodium hydroxide, sodium phosphate, dibasic, sodium phosphate, monobasic, sulphuric acid, tartaric acid, or a combination thereof.

In some embodiments, the composition can further contain an antioxidant for enhancing drug stability. The antioxidants may be selected from butylated hydroxyanisole, butylated hydroxytoluene, tert-butylhydroquinone, propyl gallate, erythorbic acid, ascorbyl palmitate, tocopherols, ethoxyquin, phosphates, ethylene diamine tetra acetic acid, tartaric acid, citric acid, lecithin, ascorbic acid, sulfites (as sulfur dioxide), ascorbyl stearate, or a combination thereof.

In some embodiments, the composition can contain a coloring agent selected from organic dyes or their lakes, natural colors, inorganic colors, or mineral colors. Dyes may be synthetic chemical compounds that exhibit certain colors. Examples of dyes may include tartrazine, erythrosine, sunset yellow, and patent blue v. lakes are aluminum salts of FD&C water-soluble dyes extended on a substratum of alumina. FD&C lakes are largely water-insoluble forms of the common synthetic water-soluble dyes and are available in six basic colors: one yellow, one orange, two reds (a pink-red and an orange-red), two blues (a green-blue and a royal blue). They can be blended to create more lake colors as needed, including brown, green, orange, red, yellow, and purple. Examples may include aluminum lakes, brilliant blue lake, sunset yellow lake, amaranth lake, allura red lake, indigo carmine lake, and quinoline yellow lake. Examples of natural colors or vegetable and animal colors are caramel, cochineal (a dried insect), carmine (the aluminum lake of the coloring matter of cochineal), riboflavin and anthocyanins, paprika oleoresin, beet root red, annatto, and curcumin. Examples of inorganic or mineral colors may include red and yellow ferric oxides and titanium dioxide.

### Aqueous Diluents

In some embodiments, the diluent used to reconstitute the freeze-dried amlodipine composition for oral solution is aqueous and free from organic solvents. The diluent can be water, juice, a buffer, or a solution. The water can be tap water, purified water, and any edible water that can be used for aiding ingestion of medication by a subject. The juice can be any juice made from fruits or vegetables that commonly exists in a human diet. The buffer and solution refer to any aqueous solution that contains buffering agents, flavoring agents, and sweeteners, or a combination thereof, or commercially available medication flavoring syrup such as Ora-Sweet®.

In some embodiments, the volume suitable for reconstituting the freeze-dried amlodipine composition for oral solution may range from 0.2 mL-240 mL (e.g., 0.2, 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240 mL). In some embodiments, the volume suitable for reconstituting the freeze-dried amlodipine composition for oral solution may be in a 1 mL-150 mL (e.g., 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150 mL) range. In some embodiments, the volume used for reconstituting the freeze-dried amlodipine composition for oral solution may be in a 2 mL-10 mL (e.g., 2, 3, 4, 5, 6, 7, 8, 9, 10 mL) range. In some embodiments, the aqueous medium has a volume of from about 0.3 mL to about 30 mL per unit dose of amlodipine base.

Oral Solutions Reconstituted from the Freeze-Dried Composition

Also disclosed herein is a reconstituted oral solution instantly produced by mixing the stable freeze-dried amlodipine composition with a reconstitution liquid (also referred to as aqueous medium, aqueous diluent). In some embodiments, the reconstituted oral solution may include an amlodipine salt in an amount corresponding to 0.0087% w/w to 10.52% w/w (e.g., 0.0087%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.1%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 10.52% w/w) of the oral solution.

In some embodiments, the reconstituted oral solution may include an amlodipine salt in an amount that delivers 0.5 mg/mL to 4 mg/mL (e.g., 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.10, 2.15, 2.20, 2.25, 2.30, 2.35, 2.4, 2.45, 2.5, 2.55, 2.6, 2.65, 2.7, 2.75, 2.8, 2.85, 2.9, 2.95, 3, 3.05, 3.10, 3.15, 3.20, 3.25, 3.30, 3.35, 3.4, 3.45, 3.5, 3.55, 3.6, 3.65, 3.7, 3.75, 3.8, 3.85, 3.9, 3.95, 4 mg/mL) of amlodipine free base.

In some embodiments, the reconstituted oral solution does not contain preservatives. In some embodiments, the reconstituted oral solution does not contain alcohol. In some embodiments, the reconstituted oral solution does not contain surfactants. In some embodiments, the reconstituted oral solution does not contain antifoaming agents. In some embodiments, the reconstituted oral solution does not contain antioxidants. In some embodiments, the reconstituted oral solution does not contain suspension aids. In some embodiments, the reconstituted oral solution does not contain flavoring agents. In some embodiments, the reconstituted oral solution does not contain coloring agents.

### Products Containing the Freeze-Dried Composition

In some embodiments, the freeze-dried amlodipine composition may be contained in one or more unit doses. As used herein, the terms “dose unit,” “unit dose,” and “dosage unit” refer to a portion of a composition that contains an effective amount of an active suitable for a single administration to provide, or contribute to, a therapeutic effect. Such dosage units may be administered one to a plurality (i.e., 1 to about 10, 1 to 8, 1 to 6, 1 to 4 or 1 to 2) of times per day, or as many times as needed to elicit a therapeutic response.

In some embodiments, one or more unit doses may be contained in one or more containers. In some embodiments, the freeze-dried amlodipine composition for an oral solution can be packaged in a container that is capable of protecting the composition from moisture and/or oxygen. In some embodiments, the container can be a bottle, packet, bag, pouch, vial, or cup in a suitable shape and size according to the quantity of the packaged composition. In some embodiments, the container can be in a combination of bottle and bag/pouch, e.g., bottles packed in bags or pouches. In some embodiments, the material made of the container can be glass, plastic, laminated aluminum, or aluminum. In some embodiments, the material made of the container can be clear glass or colored glass, e.g., amber glass. In some embodiments, the main material made of the container can be plastic material selected from high-density polyethylene (HDPE), low-density polyethylene (LDPE), polypropylene (PP), polyethylene terephthalate (PET), polycarbonate (PC), polyvinyl chloride (PVC), Polyvinylidene chloride (PVDC), Polychlorotrifluoroethylene (PCTFE), Cyclic olefin polymers (COP), or in combination thereof in a duplex or triplex laminated structure. In some embodiments, the freeze-dried amlodipine composition for oral solution is packaged in multi-dose containers. For example, multiple doses required for one course of treatment can be packaged in a suitably sized multi-dose container. In some embodiments, the freeze-dried amlodipine composition for oral solution is packaged in unit-dose containers for delivery of a single dose of amlodipine to patients at the time of administration.

In some embodiments, the size of the bottle or cup containers meets certain requirements. For example, the volume of the container needs to be big enough for holding not only the multi-dose or single-dose freeze-dried amlodipine composition but also diluent(s) for the reconstitution to happen.

The containers are further sealed with a heat induction seal or a cap. In some embodiments, the cap is a child-resistant cap.

In some embodiments, nitrogen gas may be used to purge the headspace above the freeze-dried amlodipine composition for oral solution in the container to reduce the oxygen content in the headspace to less than 18%, 15%, 12%, 8%, 4%, or 2%.

In some embodiments, the multi-dose or unit-dose containers may be further packaged in cartons, bags, or pouches for additional protection or product identification purposes. In some embodiments, the material made of these secondary packaging components can be paper, plastic, aluminum, or laminated aluminum. In some embodiments, the secondary packaging components reduce the exposure of the freeze-dried amlodipine composition for oral solution to light. In some embodiments, the secondary packaging components minimize the transmission of moisture into the freeze-dried amlodipine composition for oral solution. In some embodiments, the secondary packaging components reduce the transmission of oxygen into the freeze-dried amlodipine composition for oral solution. In some embodiments, the secondary packaging components may include product labeling information for the freeze-dried amlodipine composition for oral solution.

### Methods of Making Freeze-Dried Compositions

This disclosure also provides a process for preparing the freeze-dried amlodipine composition for oral solution. In some embodiments, the process may include: (a) dissolving an amlodipine salt and other ingredients in water to produce a clear solution; (b) freeze-drying the clear solution into a dry material in a freeze dryer; (c) filling the freeze-dried material into a container; and (d) sealing and labeling the container.

In some embodiments, the process may include: (i) dissolving an amlodipine salt and other ingredients in water to produce a clear solution; (ii) filling the solution into a container; (iii) freezing and drying the filled container in a freeze-dryer; and (iv) sealing and labeling the containers.

In the process when the amlodipine solution is freeze-dried before filling into a container, the containers can be of any material made in a bottle, packet, bag, vial, or cup form. In the process when the amlodipine solution is freeze-dried after the solution is filled into a container, the containers should be of a material made and form that is suitable for a freeze-drying process without compromising the drying effect and the integrity of the container. In some embodiments, the process produces a freeze-dried composition containing less than 5% w/w (e.g., less than 1%, 2%, 3%, 4%, 5% w/w) water.

In some embodiments, the method comprises: dissolving a therapeutically effective amount of an amlodipine salt and at least one pharmaceutically acceptable excipient to yield a solution containing from about 0.0083% w/w to 10.52% w/w (e.g., 0.0083, 0.01, 0.05, 0.1, 0.3, 0.5, 0.7, 0.9, 1.1, 1.3, 1.5, 1.7, 1.9, 2.1, 2.3, 2.5, 2.7, 2.9, 3.1, 3.3, 3.5, 3.7, 3.9, 4.1, 4.3, 4.5, 4.7, 4.9, 5.1, 5.3, 5.5, 5.7, 5.9, 6.1, 6.3, 6.5, 6.7, 6.9, 7.1, 7.3, 7.5, 7.7, 7.9, 8.1, 8.3, 8.5, 8.7, 8.9, 9.1, 9.3, 9.5, 9.7, 9.9, 10.1, 10.3, 10.5, 10.52% w/w) of the amlodipine salt; filling the solution of a single dose of the amlodipine salt into a container; freeze-drying the solution to form a freeze-dried composition containing the amlodipine salt inside the container; and sealing the container.

In some embodiments, an amlodipine salt and other ingredients may be dissolved in water to produce a bulk solution. An aliquot of the bulk solution containing a single therapeutic dose of amlodipine is filled into a container. In some embodiments, the filled containers may be loaded into a freeze dryer and are freeze-dried to form the freeze-dried amlodipine composition for oral solution. In some embodiments, the container may be sealed for protection and labeled with product information.

In some embodiments, an amlodipine salt and other ingredients may be dissolved in water to produce a bulk solution. The bulk solution is poured onto trays. The trays are loaded into a freeze dryer and freeze-dried into a bulk dry material. A portion of the bulk dry material containing single or multiple therapeutic doses of amlodipine is filled into a container. The container is then sealed for protection and labeled with product information.

### Methods of Use of Freeze-Dried Compositions

This disclosure also provides a method of treating hypertension or coronary artery disease (CAD) in a subject. CAD affects millions of Americans with a substantial impact on survival and quality of life. CAD, also known as ischemic heart disease (IHD), is a condition that affects the supply of blood to the heart. CAD is most commonly due to atherosclerotic occlusion of the coronary arteries and damage to the walls of the microvasculature.

In some embodiments, the method may include administering to that subject an oral solution instantly produced by reconstituting the freeze-dried amlodipine composition with an aqueous diluent. In some embodiments, the freeze-dried amlodipine composition may include an amlodipine salt in an amount that delivers 0.5 mg to 20 mg (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 mg) of amlodipine freebase therapeutic dose required to have a therapeutic effect.

In some embodiments, the packaged freeze-dried amlodipine composition for oral solution is projected to be stable at 15°-25° C. (59°-77° F.), such as 20°-25° C. (68°-77° F.), for at least 12-24 months, extrapolated from the stability performance of the composition stored at 40±2° C. (75% relative humidity) for at least 6 months. In some embodiments of a method of treating hypertension or CAD, a unit-dose packaged freeze-dried amlodipine composition for oral solution is used. The unit-dose packaged freeze-dried amlodipine composition for oral solution is stable at 15°-25° C. (59°-77° F.), such as 20°-25° C. (68°-77° F.), for at least 12-24 months (e.g., at least 12, 13, 14, 15, 16, 17, 18, 19, 20, 21, 22, 23, 24 months), extrapolated from the stability performance of the composition stored at 40±2° C. (75% relative humidity) for at least 6 months.

In some embodiments, each unit-dose packaged freeze-dried amlodipine composition for oral solution contains a single dose of 2.5 mg, 3 mg, 3.5 mg, 4 mg, 4.5 mg, 5 mg, 5.5 mg, 6, 6.5 mg, 7 mg, 7.5 mg, 8 mg, 8.5 mg, 9 mg, 9.5 mg, or 10 mg amlodipine base equivalent amount of amlodipine salt and does not need to be reconstituted at a pharmacy. At the time of administration, one unit-dose packaged freeze-dried amlodipine composition for oral solution is opened, and a reconstitution diluent is added to the unit-dose container. An amlodipine oral solution containing a single therapeutic dose is formed almost instantly. The amlodipine oral solution is consumed right away without the need for an in-use period.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The expression “dosage preparation” refers to the form or context in which a formulation is stored and/or used prior to or during administration to a subject. For example, a “dosage preparation” containing a formulation may constitute or comprise the formulation in the context of a vial appropriate for storage and/or administration. A dosage preparation may constitute or comprise a formulation in the context of a container that protects the formulation from light (e.g., UV light). Alternatively, a dosage preparation may constitute or comprise a formulation in the context of a container which does not protect the formulation from exposure to light.

The term “dry powder formulation” or “dry powder composition” refers to a dry, solid composition and encompasses dried compositions prepared by freeze-drying (e.g., lyophilization) or other appropriate methods (e.g., spray drying, supercritical fluid formation, etc.) to achieve production of a dried amorphous cake form. Lyophilization is a process of freeze-drying in which water is sublimed from the product after it is frozen, optionally by applying a vacuum. Specifics of lyophilizing or freeze-drying are known in the art and described, for example, in Remington's Pharmaceutical Sciences, Chapter 84, page 1565, 18th Edition, A. R. Gennaro, Editor, 1990, Mack Publishing Company. Techniques other than lyophilization which may also be used for preparation of dry powder formulation(s) (e.g., dried samples), and particularly for preparation of amorphous dry powder formulations, are known in the art, include, but are not limited to, sterile powder filling of the components, singly, or as a complete blend, spray drying, tray drying, sizing processes including milling and/or screening and precipitation. In certain embodiments, inventive dry powder formulations are in the form of a cake (e.g., an amorphous cake).

As used herein, an “effective amount” of a compound or pharmaceutically acceptable formulation can achieve a desired therapeutic and/or prophylactic effect. In some embodiments, an “effective amount” is at least a minimal amount of a compound, or formulation containing a compound, which is sufficient for treating one or more symptoms of a disorder or condition.

The term “formulation,” in general, refers to a preparation that includes at least one pharmaceutically active compound optionally in combination with one or more excipients or other pharmaceutical additives for administration to a subject. In general, particular excipients and/or other pharmaceutical additives are typically selected with the aim of enabling a desired stability, release, distribution, and activity of active compound(s) for applications.

In general, pharmaceutically acceptable salts include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, carbonate, ascorbate, succinate, maleate, gentisinate, fumarate, gluconate, glucuronate, saccharate, formate, carboxylate, benzoate, glutamate, sulfonate, methanesulfonate, ethanesulfonate, benzensulfonate, p-toluenesulfonate, selenate, and pamoate (i.e., 1,1′-methylene-bis-(2-hydroxy-3-naphthoate)) salts of compounds.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), Chinese Pharmacopoeia (ChP) etc.). Pharmaceutical grade further incorporates suitability for administration by means including topical, ocular, parenteral, nasal, pulmonary tract, mucosal, vaginal, rectal, intravenous, and the like.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Examples are provided below to further demonstrate the important aspects of the present disclosure, but it should be understood that the disclosure is not limited thereto.

### Example 1 Preparation of Amlodipine Formulation for Oral Solution

An amlodipine formulation containing 5 mg of amlodipine base was prepared according to the principles of the present disclosure. An example composition is provided in Table 1.

TABLE 1
An example freeze-dried amlodipine composition
for an oral solution containing sweetener

| | Formulation 1 |
|---|---|
| Ingredient | % by weight |
| Amlodipine Besylate | 5.24 |
| Mannitol | 94.45 |
| Neotame | 0.3 |
| Purified Water | Used but removed during processing |
| Total | 100.00 |

Mannitol and neotame were dissolved in water in a stainless steel container under stirring to make a clear solution (Solution 1). Amlodipine besylate was then slowly added to Solution 1 under stirring, and mixing was continued until amlodipine besylate was fully dissolved to obtain a clear solution (Solution 2). About 3 mL of Solution 2 was added into 10-20 mL glass or HDPE bottles. The filled bottles were loaded into a freeze-dryer. The solution-filled bottles were frozen to −40° C. Once the solution was fully converted to solid, a vacuum of 0.2 mbar was applied to initiate the primary drying process. Once the primary drying was completed, the dryer temperature was raised to 38° C. until the amount of water was below about 1%. Once the drying endpoint was reached, vacuum was released by filling nitrogen gas into the freeze-dryer. The dried bottles were unloaded, capped, and sealed under nitrogen gas protection. The sealed bottles were labeled and bundled or pouched.

### Example 2 Preparation of Amlodipine Formulation for Oral Solution

An amlodipine formulation containing 5 mg of amlodipine base, sweetener, and flavoring agent was prepared according to the principles of the present disclosure. The example composition is provided in Table 2.

TABLE 2
An example freeze-dried amlodipine composition for an
oral solution containing sweetener and flavoring agents

| | Formulation 2 |
|---|---|
| Ingredient | % by weight |
| Amlodipine Besylate | 20.64 |
| Mannitol | 76.23 |
| Sucralose | 2.98 |
| Natural mango flavor | 0.15 |
| Purified water | Used but removed during processing |
| Total | 100.00 |

Mannitol, neotame, and natural mango flavor were dissolved in water in a stainless steel container under stirring to make a clear solution (Solution 1). Amlodipine besylate was then slowly added to Solution 1 under stirring, and mixing was continued until amlodipine besylate was fully dissolved to obtain a clear solution (Solution 2). About 2.5 mL of Solution 2 was added into 20 mL glass bottles. The filled bottles were loaded into a freeze-dryer. The solution-filled bottles were frozen to −49° C. Once the solution was fully converted to solid, a vacuum of 0.12-0.22 mbar was applied to initiate the drying process. The freeze-drying process was continued to allow the product temperature to rise gradually to about 25° C. Once the drying endpoint was reached, the vacuum was released by filling nitrogen gas into the freeze-dryer. The dried bottles were removed from the freeze-dryer, capped with a rubber stopper, and sealed with an aluminum cap.

### Example 2 Quick Reconstitution

Formulations prepared according to Example 1 were reconstituted to produce amlodipine oral solutions. A bottle was opened by removing the cap/seal. About 5 mL water was added to the bottle, the time for the formulations to fully disperse and fully dissolve was recorded. The results are summarized in Table 3.

TABLE 3
Reconstitution time

| | Dispersion | Fully |
|---|---|---|
| | Time | Dissolved Time |
| Formulation | (Second) | (Second) |
| 20220501 | Instantly | <60 |
| (10 mg amlodipine per dose) | | |
| 20220402 | Instantly | <60 |
| (10 mg amlodipine per dose) | | |
| 20220401 | Instantly | <60 |
| (5 mg amlodipine per dose) | | |
| 20220601 | Instantly | <60 |
| (5 mg amlodipine per dose) | | |
| 20220604 | Instantly | <60 |
| (5 mg amlodipine per dose) | | |
| 1041-70 | Instantly | <60 |
| (2.5 mg amlodipine per dose) | | |
| 1041-72 | Instantly | <60 |
| (2.5 mg amlodipine per dose) | | |

### Example 3 Stability Characteristics

A formulation containing 5 mg amlodipine prepared according to Example 1 was stored under accelerated storage conditions at 40° C.±2° C./75% RH±5% RH, according to International Council for Harmonization (ICH) guideline Q1A, Stability Testing of New Drug Substances and Products. Samples were pulled at 1-month, 3-month, and 6-month time points, and tested for assay, impurity 2-(2-aminoethoxymethyl)-4-(2-chlorophenyl)-6-methyl-3,5-pyridinedicarboxylate fumarate (also referred to by United States Pharmacopeia as amlodipine related compound A, RC-A), unspecified unknown impurities, and total impurities. These tests were performed by using a high-performance liquid chromatographic (HPLC) method. The method uses a Waters HPLC system equipped with a UV detector, a reverse phase C8 column, an ammonium dihydrogen orthophosphate buffer, and an acetonitrile mixture as a mobile phase running at a gradient from 70:30 to 30:70 over 45 minutes.

In addition, the fast reconstitution characteristics of the formulation were also evaluated by observing the dispersing time and complete dissolving time according to the method described in Example 2.

The results are shown in Table 4. The results indicate that the formulation prepared according to Example 1 is stable after 6 months stored under the accelerated storage conditions at 40° C.±2° C./75% RH±5% RH, meeting the limits of not less than 90% of 5 mg amlodipine in the assay, not more than 1.0% of RC-A, not more than 0.2% of any unknown impurities, and not more than 1.5% of total impurities. Based on extrapolation of 6-month accelerated stability data, the amlodipine formulation is highly likely to be stable at room temperature for at least 12-24 months (e.g., at least 12, 13, 14, 15, 16, 17, 18, 19, 20, 21, 22, 23, 24 months). In addition, the formulation retained its fast reconstitution characteristics after stability.

TABLE 4
Stability of freeze-dried amlodipine compositions for oral solution

| | | | Largest | Total | Dispersing | Dissolving |
|---|---|---|---|---|---|---|
| | Assay | RC-A | unknown | impurities | Time | Time |
| Formulation | % | % | Impurity % | % | (Second) | (Second) |
| B-CIB-003 | | | | | | |
| T0 | 97.0 | 0.17 | 0.05 | 0.26 | Instantly | <60 |
| 1 Mo | 97.6 | 0.07 | 0.06 | 0.27 | Instantly | <60 |
| 3 Mo | 97.8 | 0.10 | 0.07 | 0.33 | Instantly | <60 |
| 6 Mo | 96.4 | 0.30 | 0.07 | 0.49 | Instantly | <60 |

### Example 4 Pharmacokinetic Profile & Comparison with Norvasc® Tablet

A formulation prepared according to Example 1 was found to generate a pharmacokinetic profile that is bioequivalent to that of the commercial product Norvasc® tablet when administered to human subjects at the same dose strength.

A relative bioavailability study was conducted to compare the Amlodipine Besylate for Oral Solution (test product) according to the present disclosure and commercial amlodipine tablet product (Norvasc®) (Reference product) in twelve healthy adult subjects. The study was a single dose, randomized, open-label, crossover design under fasting conditions. Each subject was dosed with a single 5 mg of either Amlodipine Besylate for Oral Solution or Norvasc® tablet after an overnight housed fasting. The test product was reconstituted using 5 mL water to produce an oral solution before dosing. Each product was dosed with 240 mL of drinking water according to a predetermined randomization table.

In each period, 18 venous blood samples were collected by using an intravenous cannula at 0 hour (within 60 mins prior to drug administration) and at 1 h, 2 h, 3 h, 4 h, 5 h, 6 h, 7 h, 8 h, 10 h, 12 h, 14 h, 24 h, 48 h, 72 h, 96 h, 120 h, 144 h after dosing.

At each sampling point, 4 mL of venous blood samples were collected and processed to separate plasma. The plasma samples were analyzed by LC-MS/MS using a validated method. The amlodipine concentrations in plasma were determined. The amlodipine concentrations were used to construct the plasma concentration vs. time profile. The pharmacokinetic parameters, i.e., including: Cₘₐₓ, AUC₀₋ₜ, and AUC_{0-∞} were calculated from the constructed profile by Phoenix WinNonlin® (Version 8.0).

For pharmacokinetics parameters Cₘₐₓ, AUC₀₋ₜ, AUC_{0-∞}, Tₘₐₓ, the arithmetic mean, standard deviation, coefficient of variation, maximum, minimum, and geometric mean of each parameter were calculated. Pharmacokinetic parameters (Cₘₐₓ, AUC₀₋ₜ, AUC_{0-∞}) from test and reference products were analyzed by variance analysis after logarithmic transform. The 90% confidence interval of the geometric mean ratio of the main pharmacokinetic parameters of amlodipine was calculated. The results are provided in Table 5. The statistical comparison shows that the geometric mean ratios of peak plasma concertation Cₘₐₓ and area under the curve AUC₀₋ₜ & AUC_{0-∞} for Amlodipine Besylate for Oral Solution and Norvasc® are 95.26%, 100.96%, and 99.90% respectively. Their 90% Confidence Intervals (CI) are within the 80.0% and 125.0% range. According to US FDA's criteria for establishing bioequivalence, Amlodipine Besylate for Oral Solution (5 mg) and Norvasc® Tablet (5 mg) are considered bioequivalent.

TABLE 5
Statistical Analysis of Amlodipine Besylate for Oral Solution (Test) versus
Norvasc ® (Reference) for Amlodipine under fasting conditions

| | Amlodipine | Norvasc ® | Geometric | | | |
|---|---|---|---|---|---|---|
| | Besylate for | Tablet, | Mean | | | |
| | Oral Solution, | 5 mg | Ratio | | | Power |
| Parameter | 5 mg (Test) | (Reference) | T/R (%) | 90% CI | ISCV % | (%) |
| Cₘₐₓ | 3.54 | 3.71 | 95.26 | 84.01~108.03 | 16.4% | 90.5% |
| (ng/mL) | | | | | | |
| AUC₀₋ₜ | 170.1 | 168.5 | 100.96 | 92.73~109.92 | 10.9% | 99.2% |
| (h*ng/mL) | | | | | | |
| AUC_{0-∞} | 186.8 | 186.9 | 99.90 | 91.53~109.04 | 11.3% | 99.0% |
| (h*ng/mL) | | | | | | |

### Example 5 Food Effect on Pharmacokinetic Profile

The formulation prepared according to Example 1 was also found to generate a pharmacokinetic profile that is not statistically changed when co-administered with food, i.e., lack of a “food effect.” A bioavailability study was conducted in twelve healthy adult subjects to compare the pharmacokinetic profiles of Amlodipine Besylate Powder for Oral Solution (containing 5 mg amlodipine) administered with and without food.

The study was in a single dose, open-label, crossover design. Subjects were dosed with a single 5 mg of Amlodipine Besylate for Oral Solution in each period under either fasting or fed conditions. The Amlodipine Besylate for Oral Solution was reconstituted using 5 mL of water to produce an oral solution before dosing. Each dose administration was dosed with 240 mL of drinking water in total volume.

In each period, 18 venous blood samples were collected by using an intravenous cannula at 0 hour (within 60 mins prior to drug administration) and at 1 h, 2 h, 3 h, 4 h, 5 h, 6 h, 7 h, 8 h, 10 h, 12 h, 14 h, 24 h, 48 h, 72 h, 96 h, 120 h, 144 h after dosing.

At each sampling point, 4 mL venous blood samples were collected and processed to separate plasma. The plasma samples were analyzed by LC-MS/MS using a validated method. The amlodipine concentrations in plasma were determined. The amlodipine concentrations were used to construct the plasma concentration vs. time profile. The pharmacokinetic parameters, i.e., including: Cₘₐₓ, AUC₀₋ₜ, and AUC_{0-∞} were calculated from the constructed profile by Phoenix WinNonlin® (Version 8.0).

For pharmacokinetics parameters, Cₘₐₓ, AUC₀₋ₜ, AUC_{0-∞}, Tₘₐₓ, the arithmetic mean, standard deviation, coefficient of variation, maximum, minimum, and geometric mean of each parameter were calculated. Pharmacokinetic parameters (Cₘₐₓ, AUC₀₋ₜ, AUC_{0-∞}) from fasting and fed periods were analyzed by variance analysis after logarithmic transform. The 90% confidence interval of the geometric mean ratio of the main pharmacokinetic parameters of amlodipine was calculated. The results are provided in Table 6. The statistical comparison shows that the geometric mean ratios of peak plasma concertation Cₘₐₓ and area under the curve AUC₀₋ₜ & AUC_{0-∞} between fasting and fed conditions are 104.97%, 97.04%, and 95.77%, respectively. Their 90% Confidence Intervals (CI) are within the 80.0% and 125.0% range. The results show that the pharmacokinetic parameters produced by the Amlodipine Besylate for Oral Solution (5 mg) under fasting and fed conditions are the same according to US FDA's criteria for establishing bioequivalence. Therefore, the amlodipine for Oral Solution formulation prepared according to the present disclosure is free of food effects.

TABLE 6
Statistical Analysis of Amlodipine Besylate for
Oral Solution under Fasting versus Fed Conditions

| | | | Geometric | | | |
|---|---|---|---|---|---|---|
| | | | Mean | | | Power |
| Parameter | Fast | Fed | Fast/Fed(%) | 90% CI | ISCV % | (%) |
| Cₘₐₓ | 3.21 | 3.06 | 104.97 | 96.26~114.47 | 11.2% | 99.2% |
| (ng/mL) | | | | | | |
| AUC₀₋ₜ | 154.2 | 158.9 | 97.04 | 92.10~102.25 | 9.5% | 100.0% |
| (h*ng/mL) | | | | | | |
| AUC_{0-∞} | 168.0 | 175.4 | 95.77 | 90.83~100.97 | 9.7% | 100.0% |
| (h*ng/mL) | | | | | | |

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A freeze-dried composition, comprising at least one therapeutically effective amount of amlodipine salt and at least one pharmaceutically acceptable excipient, wherein the composition is adapted to form an oral solution when admixed with an aqueous medium, wherein the composition remains stable at 20-25° C. for a storage period of at least 12-24 months, and wherein the composition comprises 1.0% (wt/wt) or less of 3-ethyl 5-methyl[2-(2-aminoethoxymethyl)-4-(2-chlorophenyl)-6-methyl-3,5-pyridinedicarboxylate fumarate at the end of the 12-24 months storage period.

2. The composition of claim 1, wherein the oral solution containing 5 mg equivalent of amlodipine base following a single oral administration of the oral solution to adult subjects under fasted conditions provides the same pharmacokinetic profile as commercially available tablet containing 5 mg equivalent of amlodipine base, wherein the pharmacokinetic profile has an area under the curve of from about 83.5 ng·hr/mL to about 256.5 ng·hr/mL and a Cmax of from about 1.8 ng/ml to about 5 ng/ml.

3. The composition of claim 1, wherein the composition is capable of dispersing and dissolving within less than 60 seconds upon admixing with the aqueous medium.

4. The composition of claim 1, wherein the composition is capable of dispersing and dissolving within less than 60 seconds upon admixing with the aqueous medium, after storage at 20-25° C. for a period of at least 12-24 months.

5. The composition of claim 1, wherein the composition is in the form of power, granules, agglomerates, or power cake.

6. The composition of claim 1, wherein the composition comprises from about 0.035% (wt/wt) to about 99.64% (wt/wt) of the amlodipine salt.

7. The composition of claim 1, wherein the oral solution comprises from about 0.0087% w/w to about 10.52% w/w of the amlodipine salt.

8. The composition of claim 1, wherein the amlodipine salt comprises amlodipine besylate.

9. The composition of claim 1, wherein the composition comprises from about 0.0005% w/w to about 99.96% w/w of the excipient.

10. The composition of claim 1, wherein the excipient comprises a bulking agent.

11. The composition of claim 10, wherein the excipient comprises from about 0% w/w to about 99.96% w/w of the bulking agent.

12. The composition of claim 10, wherein the oral solution comprises from about 0% w/w to about 97.1% w/w of the bulking agent.

13. The composition of claim 1, wherein the excipient comprises a taste-masking agent.

14. The composition of claim 13, wherein the composition comprises from about 0.0005% w/w to about 99.8% w/w of the taste-masking agent.

15. The composition of claim 13, wherein the oral solution comprises from about 0.000125% w/w to about 43.13% w/w of the taste-masking agent.

16. The composition of claim 13, wherein the composition comprises:
about 5-7% w/w of the amlodipine salt;
about 91-95.9% w/w of the bulking agent; and
about 0.1-2% w/w of the taste-masking agent.

17. The composition of claim 13, wherein the oral solution comprises:
from about 0.068% w/w to about 0.26% w/w of the amlodipine salt;
from about 1.234% w/w to about 4.75% w/w of the bulking agent;
from about 0.0039% w/w to about 0.015% w/w of the taste-masking agent; and
from about 94.87% w/w to about 98.69% w/w of water.

18. The composition of claim 1, wherein the composition further comprises a coloring agent, an antioxidant, a pH-adjusting agent, an antifoaming agent, or a combination thereof.

19. The composition of claim 1, wherein the aqueous medium comprises water, juice, a buffer, a solution, or a combination thereof.

20. The composition of claim 1, wherein the aqueous medium comprises a sweetened water solution, a flavored water solution, or a combination thereof.

21. The composition of claim 1, wherein the aqueous medium has a volume of from about 0.3 mL to about 30 mL per unit dose of amlodipine base.

22. The composition of claim 1, wherein the aqueous medium is free of an organic solvent or a harmful preservative.

23. The composition of claim 1, wherein the oral solution is adapted for oral administration to a human subject who has difficulty swallowing a rigid-shaped solid pharmaceutical dosage form.

24. The composition of claim 1, wherein the composition is provided as one or more unit doses.

25. The composition of claim 1, wherein the composition is prepared by in situ freeze-drying a liquid solution of the composition in the container.

26. A unit dose comprising the composition of claim 1.

27. An oral solution for oral administration prepared by reconstituting the composition claim 1.

28. A method for forming a composition according to claim 1, comprising:
dissolving a therapeutically effective amount of an amlodipine salt and at least one pharmaceutically acceptable excipient to yield a solution containing from about 0.0087% w/w to 10.52% w/w of the amlodipine salt;
filling the solution of a single dose of the amlodipine salt into a container;
freeze-drying the solution to form a freeze-dried composition containing the amlodipine salt inside the container; and
sealing the container.

29. A method of treating hypertension or coronary artery disease (CAD) in a subject in need thereof, comprising: reconstituting a composition according to claim 1 with an aqueous medium to form an amlodipine oral solution at the time of administration, and orally administering to the subject the oral solution comprising a therapeutically effective amount of amlodipine.

30. The method of claim 29, wherein the therapeutically effective amount of amlodipine administered produces a pharmacokinetic profile bioequivalent to that of an amlodipine tablet containing 5 mg equivalent of amlodipine base in adult subjects under fasted conditions.
